(19) United States
(12) Patent Application Publication
(10) Pub. No.: US 2008/0262249 A1
(43) Pub. Date: Oct. 23, 2008

(54) Psorospermin and Analogues

(76) Inventors: Jeffrey P. Whitten, Santee, CA (US); Jing-Yu Lai, San Diego, CA (US)

Correspondence Address: MORRISON & FOERSTER LLP, 12531 HIGH BLUFF DRIVE, SUITE 100, SAN DIEGO, CA, 92130-2040, US

(21) Appl. No.: 11/660,417

(22) PCT Filed: Aug. 16, 2005
(86) PCT No.: PCT/US2005/028986

§ 371 Date: Jan. 7, 2008

Related U.S. Application Data
(60) Provisional application No. 60/602,464, filed on Aug. 17, 2004.

(51) Int. Cl. C07D 493/02

(52) U.S. Cl. 549/383

(57) ABSTRACT

Methods of making chiral psorospermin or its analogues and/or intermediates thereof are provided.

### CROSS-REFERENCE TO RELATED APPLICATIONS

This application is related to U.S. provisional application Ser. No. 60/602,464, filed 17 Aug. 2004.

### FIELD OF THE INVENTION

The invention relates to the production of chiral psorospermin and its analogues.

### BACKGROUND

Psorospermin is a novel cytotoxic dihydrofuranoxanthone isolated from the roots and stembark of the African plant Psorospermum febrifugum (Kupchan et al., J. Nat. Prod. 43, 296-301, (1980)). Psorospermin is particularly intriguing because of an apparent dilemma: low reactivity and poor sequence selectivity toward duplex DNA but much greater activity than expected in in vitro cytotoxicity assays and an even more interesting profile in the NCI 60-panel screen.

From these intriguing results it has been postulated that a selectivity trigger must exist in vitro, and this trigger could be due to a DNA-protein-drug interaction, which requires topoisomerase I or II as potential cross-linking proteins (Permana, P. et al., Cancer Res. 54, 3191-3195 (1994)).

Although the racemic psorospermin methyl ether synthesis has been reported, no chiral synthesis of the parent psorospermin has been reported (Ho, D. K., et al., J. Org. Chem. 52, 342-347 (1987); Reddy, K. S., et al., Tetrahedron Letters 28, 3075-3078 (1987)).

### SUMMARY OF THE INVENTION

The methods of the invention relate to producing psorospermin and analogues of structures 1 and II and intermediates thereof:

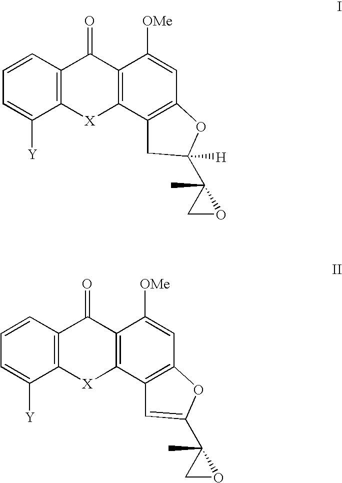

where X is O, S, NH, or NR; Y is H, OH, OR, R, Cl, F, or NHSO₂CH₃; and where R is C₁-C₁₀ hydrocarbyl.

As used herein, “hydrocarbyl” refers to a hydrocarbon residue which contains only carbon and hydrogen. The residue may be aliphatic or aromatic, straight-chain, cyclic, branched, saturated or unsaturated.

### DETAILED DESCRIPTION OF THE INVENTION

In one embodiment an intermediate is prepared by a process comprising:

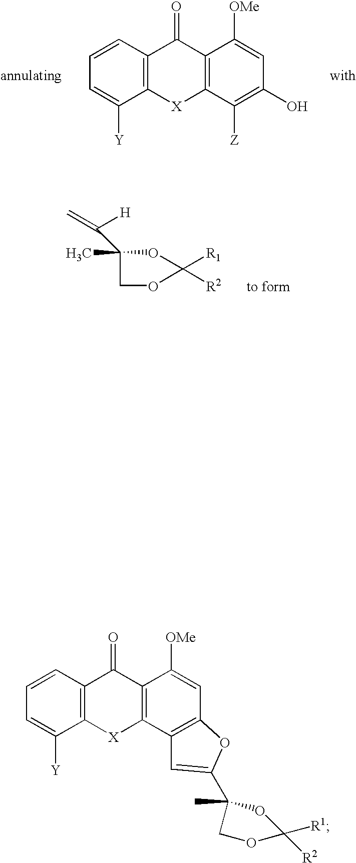

wherein X is O, S, NH, or NR;

wherein Y is H, OH, OR, R, OCOR, Cl, F, or NHSO₂CH₃;

wherein R is C₁-C₁₀ hydrocarbyl,

wherein Z is halogen; and

wherein each of R¹ and R² is independently H or C₁-C₁₀ hydrocarbyl, or R¹ and R² join together to form a C₅-C₇ ring.

As used herein, “hydrocarbyl” refers to a hydrocarbon residue which contains only carbon and hydrogen. The residue may be aliphatic or aromatic, straight-chain, cyclic, branched, saturated or unsaturated.

Preferably the annulating step of this process is performed using a transition metal catalyst.

The process for preparing an intermediate of psorospermin or an analogue thereof may further comprise, in one embodiment, before the annulating step, selectively halogenating

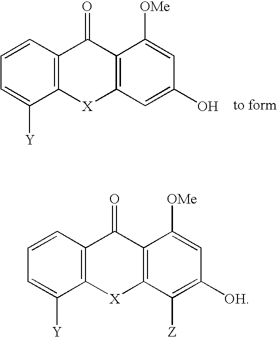

Preferably, the halogenating is performed with an elemental or complexed halogen.

In another embodiment of a process to make the intermediate, the process may further comprise before said halogenating step, condensing

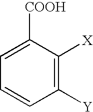

with phloroglucinol to form

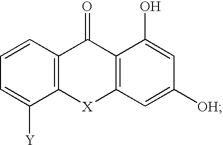

selectively protecting

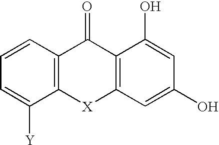

by adding benzyl halide to form

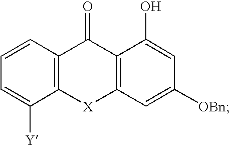

and methylating

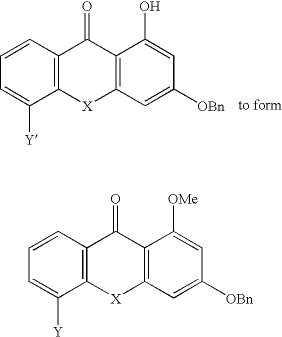

wherein Y′ is either Y or a protected OH group. Preferably the condensing step is performed in a phosphorosoxychloride solvent with a Lewis acid catalyst; wherein the protecting step is performed using benzyl halide wherein the protected Y group is O-benzyl; and the methylating step is performed using methyl iodide.

In another process for preparing an intermediate, the annulating step described above is followed by reducing

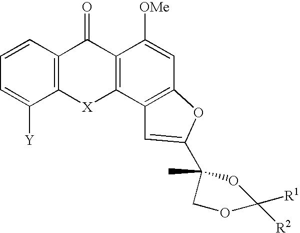

to form 2′R,3′R and 2′S,3′R diastereomers of

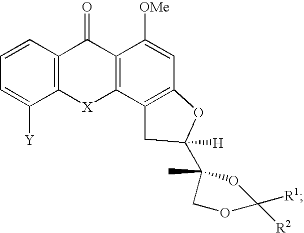

and separating the diastereomers;

wherein each of R¹ and R² is independently H or C₁-C₆ hydrocarbyl, or R¹ and R² join together to form a C₅-C₇ hydrocarbyl ring. Preferably, the reducing step is performed using hydrogen atmosphere and a transition metal catalyst; and the separating step is performed by crystallization or chromatography.

In a further embodiment, the process further comprises deprotecting at least one of the separated diastereomers.

After deprotecting at least one of the separated diastereomers, the process may further comprise cyclizing to an epoxide at least one of the separated diastereomers to form

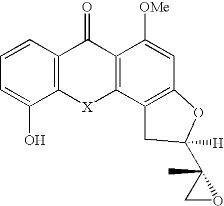

or the 2′S,3′R form thereof to form psorospermin or an analogue thereof. Preferably, the cyclizing step includes the sub steps of activating a hydroxyl group of

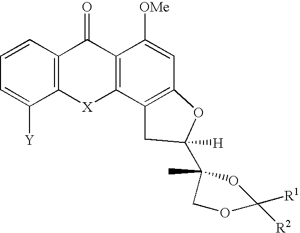

or the 2′S,3′R form thereof with a mesylate, and cyclizing under basic conditions.

In another embodiment for preparing psorospermin or an analogue thereof, the process comprise after the cyclizing step alkylating

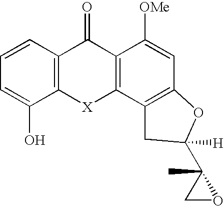

or the 2′S,3′R form thereof to form

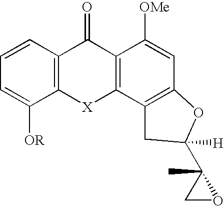

or the 2′S,3′R form thereof. Preferably, the alkylating step is performed using an alkyl halide.

The process for making psorospermin or an analogue thereof, may also comprise, in one embodiment, epoxidizing

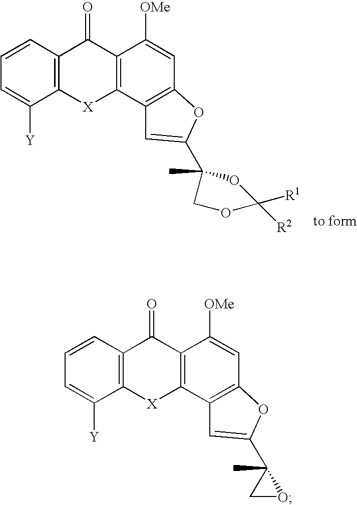

wherein each of R¹ and R² independently is H or C₁-C₆ hydrocarbyl, or R¹ and R² join together to form a C₅-C₇ hydrocarbyl ring. Preferably, the cyclizing step includes the sub steps of forming a diol on

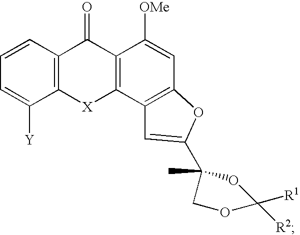

activating the hydroxyl groups of the diol with a mesylate; and cyclizing under basic conditions.

In another embodiment, the process for making psorospermin in analogues thereof comprises alkylating

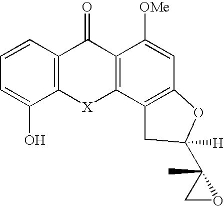

or the 2′S,3′R form thereof to form

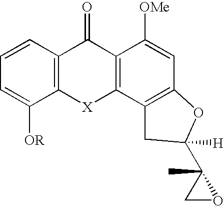

or the 2′S,3′R form thereof;

wherein X is O, S, NH, or NR; and

wherein R is C₁-C₁₀ hydrocarbyl.

In yet a further embodiment, a process for making psorospermin or an analogues thereof comprises cyclizing to an epoxide at least one of

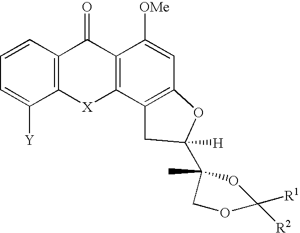

or the or 2′S,3′R form thereof to form

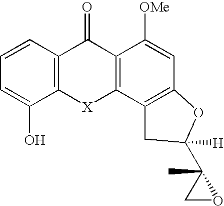

or the 2′S,3′R form thereof;

wherein each of R¹ and R² independently is H or C₁-C₆ hydrocarbyl, or R¹ and R² join together to form a C₅-C₇ hydrocarbyl ring;

wherein Y is H, OH, OR, R, Cl, F, or NHSO₂CH₃; and

wherein R is C₁-C₁₀ hydrocarbyl.

The process may also include before said cyclizing step, reducing

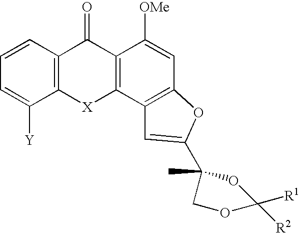

to form 2′R,3′R and 2′S,3′R diastereomers of

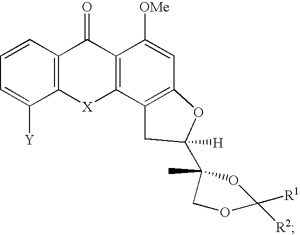

and separating the diastereomers.

The process may further comprise before the reducing step annulating

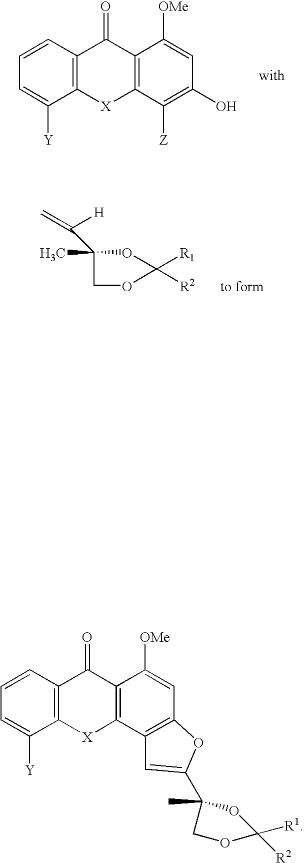

The compounds described above were prepared according to the steps involved in the following scheme:

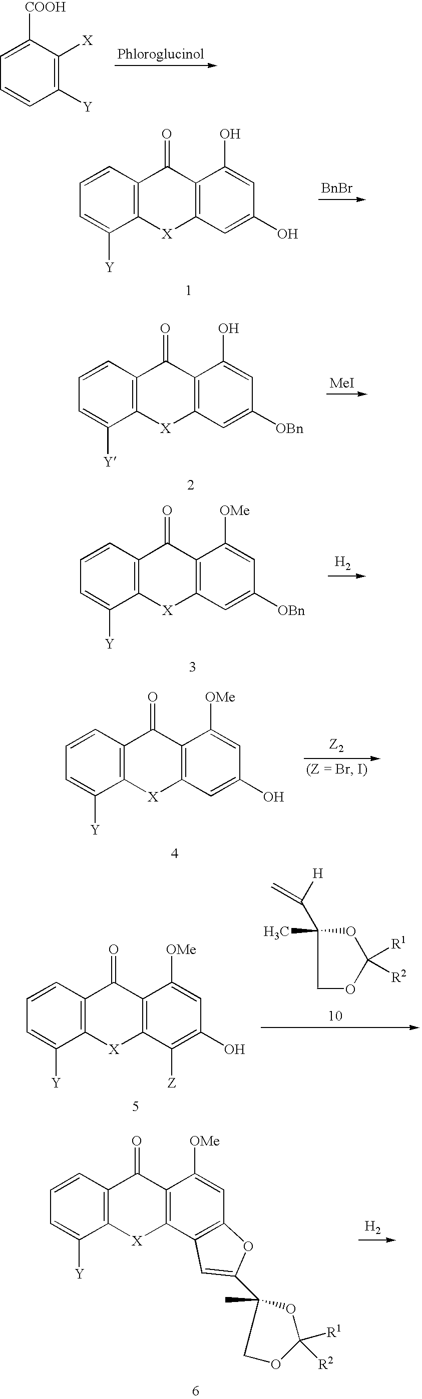

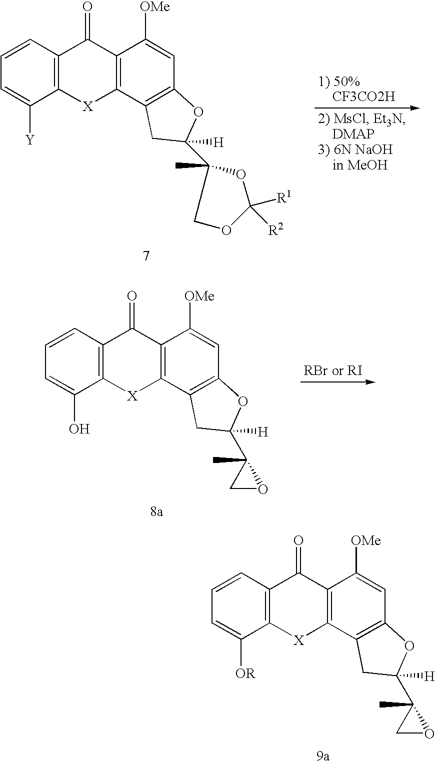

where a suitably substituted benzoic acid is heated with phloroglucinol under Fridel Craft acylation conditions to yield xanthone 1. Preferably X is O, S, NH, or NR; and Y is H, OH, OR, R, Cl, or F, where R is C₁-C₁₀ hydrocarbyl. Typically a solvent such as phosphorosoxychloride with a Lewis acid catalyst typically zinc chloride is heated between 50 and 120° C. The product 1 is then selectively protected with an aryl OH blocking group such as benzyl. Careful addition, under basic conditions, of between 2 to 10 equivalents of a benzyl halide, such as benzyl bromide, in a solvent such as acetone between 10° C. to 60° C. leads to the product 2.

1-Methoxyxanthone 3 is prepared from compound 2 by treatment with an excess of a methylating agent. Any suitable methylating agent can be used such as dimethyl sulfate, or more preferably methyl iodide. Optimal conditions are using between 1 to 10 equivalents of the methylating agent at a temperature between 25° C. to 60° C. The blocking group of Y′ is then removed by treatment under Lewis Acid conditions or in the case of benzyl with hydrogen and a transition metal catalyst such as palladium. Typical conditions are between 20° C. and 60° C. in a solvent such as ethanol, methanol, dichloromethane or acetic acid with a catalyst such as palladium or a supported catalyst such as palladium on carbon under an atmosphere of hydrogen which varies between 1 and 10 atmospheres.

The xanthone 4 is then selectively halogenated to yield alpha-halo phenol 5, wherein Z is a halogen. Typical conditions can be reaction with elemental halogens such as chlorine, bromine or iodine in inert solvents such as dichloromethane. Greater selectivity is achieved with complexed halogens such as pyridinium tribromide in an aprotic polar solvent such as DMF or NMP at a temperature between 0° C. and 50° C.

Compound 5 is then annulated in a novel one pot procedure to furan 6 using substituted alkenes and a transition metal catalyst. Preferably, R₁ and R₂ are each H or C₁-C₆ hydrocarbyl, or R¹ and R² join to form a C₅-C₇ hydrocarbyl ring, preferably a C₆ hydrocarbyl ring. The catalyst is typically palladium and can be stabilized with phosphine ligands or preferentially used as the palladium salt. Even more preferred is palladium acetate as the salt. Solvents for the reaction include aprotic solvent such as DMF or NMP at a temperature between 60° C. and 160° C.

Suitably substituted olefins yield compounds 6 that can be derivatived into natural products and their analogs. Compound 6 is reduced under an atmosphere of hydrogen and a transition metal catalyst. This catalyst can be palladium or palladium supported on an inert support such as carbon or more preferred can be activated nickel. The temperature of the reaction varies between 25 and 65° C. between one and ten atmospheres of hydrogen in a solvent such as ethanol, methanol or acetic acid. The resulting diastereomers, when a chiral substituent is present, are separated by processes well known in the art such as crystallization or chromatography to yield pure chiral annulated dihydrofurans 7.

Chiral compounds 7 are then deprotected by treatment with mild acid conditions. The acid can be a mineral acid such as dilute hydrochloric acid in water or aqueous acetone or with 50% trifluoroacetic acid in water at room temperature. Formation of the epoxide is by activation of a hydroxyl group with a derivative such as a halogen or preferentially by formation of the mesylate which upon base treatment cyclizes to epoxide 8a or the 2′S,3′R form thereof. Preferred conditions for cyclization are with sodium hydroxide in an alcohol solvent such as methanol at room temperature.

Compound 8a or the 2′S, 3′R form thereof can then be alkylated with alkyl iodides to give compounds 9a or the 2′S, 3′R form thereof which may have superior biological properties over the parent compound.

### Relationship Between Psorospermin and DNA Topoisomerase II

(a) Structure of the psorospermin-(N7-guanine)-DNA adduct. In the initial study, Hansen et al, J. Am. Chem. Soc. 118, 5553-5561 (1996), used gel electrophoresis and high-field NMR to define a mechanism for covalent reaction of psorospermin with N7 of guanine in DNA and to determine the DNA sequence selectivity for this covalent reaction (Hansen, M., et al., (1996), supra). First, psorospermin is between 10¹ and 10² less reactive toward duplex DNA than the structurally similar antibiotics the pluramycins. Also, unlike the pluramycins there is no selectivity for the base pair to the 3′ side of the alkylated guanine, but there is a distinct selectivity for the base pair to the 5′ side. For both high- and medium-reactivity sites, psorospermin shows the greatest preference for a guanine located to the 5′ side, a second preference for an adenine in the 5′ position, and only low reactivity with guanines having a pyrimidine at the same position. Psorospermin intercalates into the DNA and positions the reactive epoxide into the proximity of the guanine that is located to the 3′ side of the intercalation site.

NMR results indicate that covalent attachment occurs between N7 of guanine and C4′ of the epoxide on the psorospermin ligand. However, despite these similarities, the proposed precovalent mode of DNA binding is more similar to the acridine class of agents than to the pluramycins (Hansen et al., supra). Like the acridines, psorospermin stacks its aromatic chromophore in an orientation parallel to the adjoining base pairs, as opposed to an orthogonal orientation characteristic of the pluramycins (Hansen and Hurley, J. Am Chem. Soc. 117, 2421-2429 (1995)); Hansen et al., (1996) supra; Sun, D., et al., J. Am. Chem. Soc. 117, 2430-2440 (1995)) (FIG. 4). In this respect, the psorospermin-DNA interaction resembles that of the quinacrine nitrogen mustard (Baguley, B., Anti-Cancer Drug Des. 6, 1-35 (1991); Gopalakrishnan, S. et al., Biochemistry 31, 10790-10801 (1992)). This parallel, as opposed to orthogonal, orientation to the base pairs is important because it reinforces the idea that maximizing base-stacking interactions is critical for stabilization of the complex prior to covalent alkylation in the absence of significant groove interactions. Furthermore, even with these enhanced base-pair stacking interactions, psorospermin has only a modest to poor alkylation ability. This is important because the alkylation sequence selectivity is determined by a site-directed alkylation by topoisomerase II (see below), and in order to achieve maximum selectivity, the covalent reactivity in the absence of topoisomerase II should be minimal.

(b) Topoisomerase II directs site-directed alkylation of DNA by psorospermin. The key observation with psorospermin is that topoisomerase II directs the sequence-specific alkylation of DNA by psorospermin, while in the same experiment, pluramycin alkylation was inhibited with increasing topoisomerase II concentration. While psorospermin shows poor sequence selectivity and reactivity with DNA in a cell-free system, in in vitro systems it shows a much higher reactivity and a sequence selectivity that is directed by topoisomerase II. This is a beautiful example of how a DNA-interactive protein (topoisomerase II) can enhance the sequence selectivity of an apparently poorly selective alkylating agent. The stereochemical requirement dictates why topoisomerase II enhancement of psorospermin occurs, while pluramycin is unaffected. Because topoisomerase II greatly enhances the psorospermin alkylation of the guanine at the +4′ position of site B, it was important to determine the effect of psorospermin on the topoisomerase II-mediated DNA cleavage. In the absence of psorospermin, the intensity of the topoisomerase II-mediated DNA cleavage is much less at site B than at site A. As the concentration of psorospermin was increased, the topoisomerase II-mediated DNA cleavage at site A was decreased, while the cleavage at site B was enhanced. The psorospermin-induced DNA cleavage by topoisomerase II reaches a maximum of 3-fold at a 10 μM drug concentration. This result suggests that psorospermin alkylation at site B traps the topoisomerase II-DNA complex at this site. On the other hand, the cleaved complex formation at site A was reduced in the presence of psorospermin, despite the 3-fold enhancement of psorospermin alkylation at site A. Sites A and B are three base pairs apart from each other, and Drosophila topoisomerase II binds a region of approximately 23 base pairs, based on the results of a DNase I footprinting experiment (Lee et al., J. Biol. Chem., 264, 21779-21787 (1989)). Therefore, it is likely that sites A and B are competing with each other for topoisomerase II binding, and the 25-fold enhancement of the psorospermin alkylation at site B dominates this competition. Because psorospermin is a 7-alkyl adduct, depurination occurs slowly at room temperature over a period of several days.

(c) The topoisomerase II-induced DNA cleavage by psorospermin is reversible. In subsequent work the alkylating site within the topoisomerase II gate was defined and determined the timing when the alkylation occurs in the topoisomerase II cleavage and resealing cycle (Kwok Y. et al., J. Biol. Chem. 273, 33020-33026 (1998)). First, it was demonstrated that the topoisomerase II-induced alkylation of DNA by psorospermin occurs at a time preceding the topoisomerase II-mediated strand cleavage event because it occurs in the absence of Mg²⁺. The alkylation of DNA by psorospermin has been reported to take place at N7 of guanine in the presence of topoisomerase II since substitution of the target guanine by 7-deazaguanine prevents alkylation. Because the stimulation of the topoisomerase II-induced DNA cleavage by psorospermin can be slowly reversed by the addition of excess salt, this indicates that alkylation of DNA by psorospermin traps a reversible topoisomerase II-DNA complex. Finally, it has been suggested that it is the psorospermin-DNA adducts, not the abasic sites resulting from depurination, that are responsible for the stimulation of the topoisomerase II-mediated cleavage. Since the precise location of the psorospermin within the topoisomerase II cleavage site is known, together with the covalent DNA linkage chemistry and the conformation of the psorospermin-DNA adduct, this structural insight provides an excellent opportunity for the design and synthesis of new, more effective topoisomerase II poisons. Psorospermin has a number of intrinsic features that have apparent advantages over existing topoisomerase II poisons or sequence-specific alkylators. First, psorospermin is a covalent topoisomerase II poison and will accordingly have an infinite “dwell time” at the topoisomerase II gate in comparison to doxorubicin or mitoxanthone. Second, because of the topoisomerase II site-directed alkylation, psorospermin has much greater sequence selectivity than comparable alkylating agents.

All references cited throughout the specification are expressly incorporated herein by reference. While the present invention has been described with reference to the specific embodiments thereof, it should be understood by those skilled in the art that various changes may be made and equivalents may be substituted to adapt the present invention to a particular situation. All such changes and modification are within the scope of the present invention.

The following examples are intended to illustrate but not to limit the invention.

### EXAMPLES

### Example 1

### Preparation of Compound 1

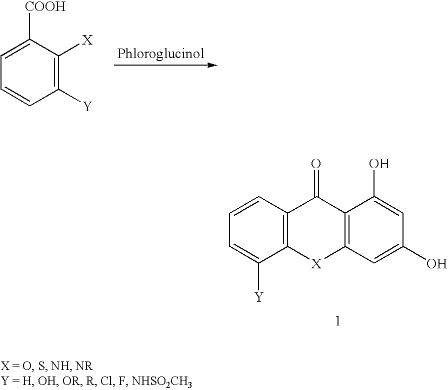

Freshly fused ZnCl₂ (35 g, 224 mmol) was added with benzoic acid (175 mmol), phloroglucinol (32 g, 253 mmol), and phosphorus oxychloride (200 mL). The mixture was stirred at 80° C. for 2 hours. After cooled to room temperature, the red oil was slowly poured onto the crushed ice (1500 g), the precipitated red solid was allowed to settle overnight, collected by filtration, air-dried, and further dried at 100° C. in vacuo for 12 hours. The crude product was dissolved in acetone (4000 mL) and refluxed for 2 hours. After cooled to room temperature, the mixture was passed through a short column packed with silica gel (6 inches thick), washed with ethyl acetate. The combined filtrate and washings were concentrated in vacuo to give a light yellow powder. When X=O and Y=OH, the yield was 10-30%. ¹H NMR (acetone-d₆) δ 12.95 (s, 1H, OH), 9.62-9.49 (br, 2H, OH), 7.66 (dd, J=7.31, 2.05 Hz, 1H), 7.33 (dd, J=8.24, 2.05 Hz, 1H), 7.26 (dd, J=7.31, 8.24 Hz, 1H), 6.46 (d, J=2.0 Hz, I H), 6.26 (d, J=2.03 Hz, 1H). MS m/e 245 (M+H⁺).

### Example 2

### Preparation of Compound 2

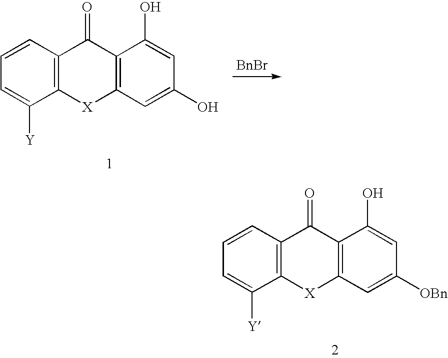

A solution of compound 1 (14 mmol), benzyl bromide (6.0 g, 35 mmol), K₂CO₃ (10 g, 72 mmol) in acetone (200 mL) was refluxed under Argon for 16 hours. After the reaction mixture was cooled to room temperature, the potassium salts were filtered off. The filtrate was concentrated in vacuo, the residue was rinse with hexanes and filtered to give an off-white powder. When X=O and Y′=OBn, the yield was 83%. ¹H NMR (CDCl₃) δ 12.86 (s, 1H, OH), 7.83 (d, J=7.29, 1.99 Hz, 1H), 7.50 (d, J=7.55 Hz, 1H), 7.49-7.40 (m, 6H), 7.40-7.30 (m, 3H), 7.28-7.20 (m, 2H), 6.63 (d, J=2.69 Hz, 1H), 6.44 (d, J=2.09 Hz, 1H), 5.37 (s, 2H), 5.15 (s, 2H).

### Example 3

### Preparation of Compound 3

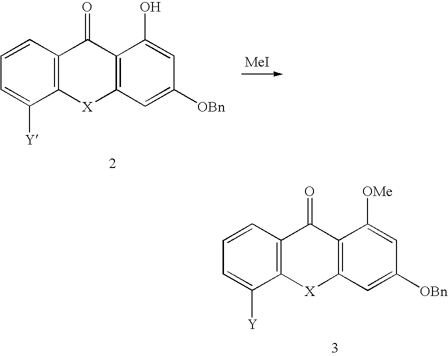

A solution of compound 2 (8.7 mmol), iodomethane (1.4 mL, 22 mmol), K₂CO₃ (10 g, 72 mmol) in acetone (200 mL) was refluxed under Argon for 16 hours. After the reaction mixture was cooled to room temperature, the potassium salts were filtered off. The filtrate was concentrated in vacuo, the residue was rinse with hexanes and filtered to give an off-white powder. When X=O and Y=OH, the yield was 85%. ¹H NMR (CDCl₃) δ 7.90-7.87 (m, 1H), 7.52-7.34 (m, 11H), 7.21-7.17 (m, 1H), 6.69 (d, J=2.09 Hz, 1H), 6.44 (d, J=2.68 Hz, 1H), 5.27 (s, 2H), 5.15 (s, 2H), 3.97 (s, 3H). MS m/e 439 (M+H⁺), 425, 348, 305, 261.

### Example 4

### Preparation of Compound 4

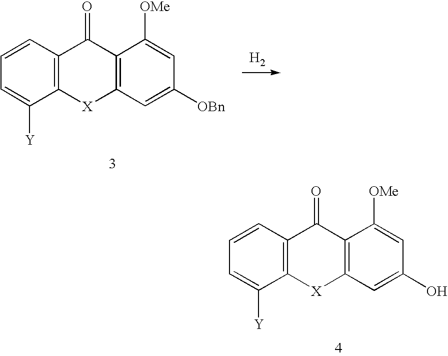

To a solution of compound 3 (9.1 mmol) in 10% methanol in dichlormethane (200 mL), Pd(OH)₂/C (170 mg) was added under Argon. The mixture was shaken at room temperature under the pressure of hydrogen (50 psi) for 15 hours, and filtered. After the collected solid was washed with DMF, the combined filtrate and washings were concentrated in vacuo to give a beige powder. When X=O and Y=OH, the yield was 86%. ¹H NMR (DMSO-d₆) δ 10.98-10.62 (br, 1H, OH), 10.30-10.04 (br, 1H, OH), 7.46 (dd, J=7.90, 1.85 Hz, 1H), 7.17 (d, J=8.01 Hz, 1H), 7.12 (dd, J=7.87, 7.86 Hz, 1H), 6.46 (d, J=1.98 Hz, 1H), 6.35 (d, J=1.90 Hz, 1H), 3.84 (s, 3H).

### Example 5

### Preparation of Compound 5

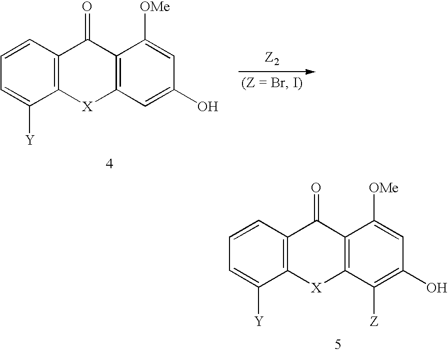

A solution of compound 4 (3.5 mmol), iodine (1.9 g, 7.5 mmol) or pyridinium tribromide (1.2 g, 3.7 mmol) in DMF (5 mL) was stirred under argon at room temperature overnight. The reaction mixture was poured into water (15 mL), followed by filtration to give a brown powder. The brown powder was stirred in cyclohexene (100 mL) for 3 hours to give a light tan color powder after filtration. When X=O and Y=OH, the yield was quantitative. ¹H NMR (DMSO-d₆) δ 11.60 (s, 1H, OH), 10.27 (s, 1H, OH), 7.45 (dd, J=7.90, 1.35 Hz, 1H), 7.24 (dd, J=7.85, 1.30 Hz, 1H), 7.18 (dd, J=7.85, 7.85 Hz, 1H), 3.84 (s, 3H). MS m/e 385 (M+H⁺), 370, 246.

### Example 6

### Preparation of Compound 6

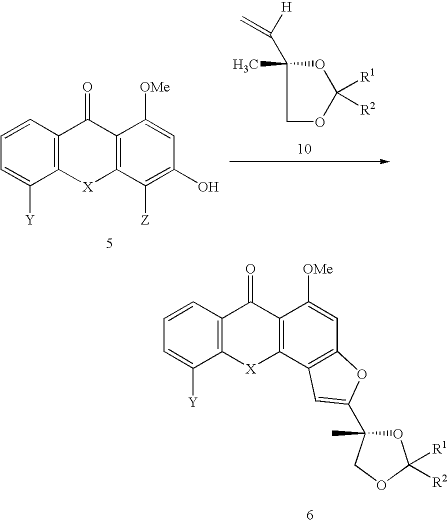

A solution of compound 5 (1.0 mmol), (3R)-compound 10 (1.6 mmol), sodium bicarbonate (338 mg, 4 mmol) and palladium acetate (44.9 mg, 0.2 mmol) in anhydrous DMF (4 mL) and dioxane (4 mL) was sealed, and then heated at 120° C. for 8-16 hours. After cooled, the mixture was added with ethyl acetate (200 mL), washed with a saturated solution of ammonium chloride (2×30 mL), brine (2×30 mL). The organic layer was dried over magnesium sulfate, concentrated, and isolated by chromatography on silica gel eluting with 20-50% acetone in hexanes, to give (3′R)-compound 6. When X=O and Y=OH, R¹ and R²=—(CH₂)₅—, the yield was 20-50%. ¹H NMR (CDCl₃) δ 7.77 (dd, J=8.14, 1.35 Hz, 1H), 7.36 (dd, J=8.18, 1.32 Hz, 1H), 7.21 (dd, J=8.11, 8.12 Hz, 1H), 7.04 (s, 1H), 6.82 (s, 1H), 4.33 (d, J=8.42 Hz, 1H), 3.98 (s, 3H), 3.96 (d, J=9.14 Hz, 1H), 1.80-1.50 (m, 8H), 1.65 (s, 3H), 1.50-1.48 (m, 1H), 1.48-1.22 (m, 1H). ¹³C NMR (CDCl₃) δ 176.36, 159.72, 159.22, 158.88, 151.26, 144.79, 143.71, 124.38, 124.11, 119.95, 117.80, 111, 86, 110.85, 108.78, 100.35, 91.47, 77.97, 73.53, 56.84, 36.74, 36.09, 25.31, 25.26, 24.15, 24.06. MS m/e 437 (M+H⁺), 423, 303, 287.

### Example 7

### Preparation of Compound 7

### 2′R, 3′R

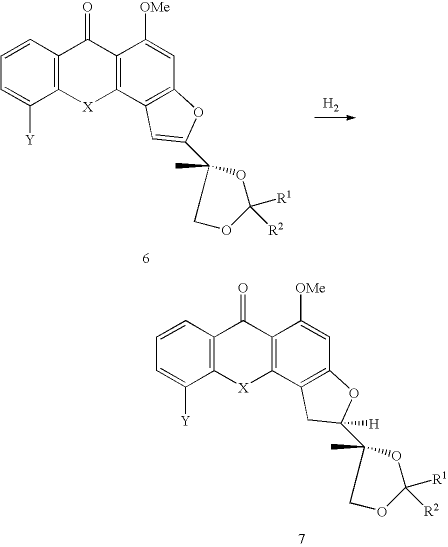

To a solution of (3′R)-compound 6 (0.0917 mmol) in ethanol (10 mL), Raney Nickel (100 mg) was added under argon. The reaction mixture was shaken at room temperature under the pressure of hydrogen (50 psi) for 8 hours. After the catalyst was filtered off, the filtrate was concentrated and purified by PTLC (40-50% acetone in hexanes), to give the (2′R,3′R)-compound 7 and (2′S, 3′R)-compound 7. When X=O and Y=OH, R¹ and R²=—(CH₂)₅—, the yield of (2′R, 3′R)-compound 7 was 44.7% ((2′S, 3′R)-compound 7 was obtained in 30.0% yield). ¹H NMR (CDCl₃) δ 7.82 (dd, J=8.37, 1.95 Hz, 1H), 7.25 (dd, J=8.14, 1.95 Hz, 1H), 7.20 (dd, J=8.14, 8.02 Hz, 1H), 6.39 (s, 1H), 4.93 (dd, J=9.22, 9.92 Hz, 1H), 4.18 (d, J=8.41 Hz, 1H), 3.96 (s, 3H), 3.84 (d, J=8.35 Hz, 1H), 3.44-3.32 (m, 2H), 1.75-1.44 (m, 8H), 1.41 (s, 3H), 1.36-1.18 (m, 2H). MS m/e 439 (M+H⁺), 349, 305.

### Example 8

### Preparation of Compound 8

### 2′R,3′R

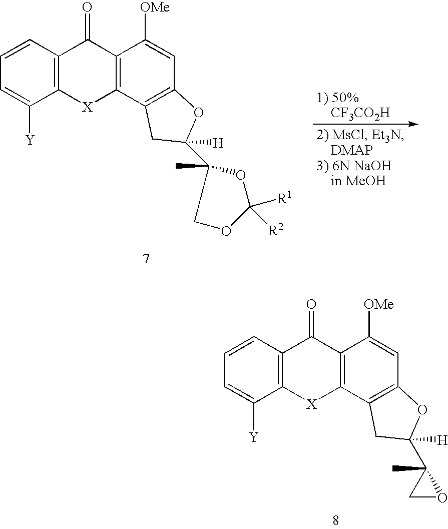

A solution of (2′R,3′R)-compound 7 (0.00923 mmol) in 50% trifluoroacetic acid in water (0.8 mL) was stirred at room temperature for 40 minutes, and then concentrated in vacuo to give the crude diol, which was used without further purification.

The crude diol and a catalytic amount of DMAP (1 mg) was dissolved in anhydrous dichloromethane (1.0 mL) and cooled to −40° C. Triethyl amine (12.7 μL, 0.0923 mmol) was added into the solution, followed by the slow addition of methanesulfonyl chloride (2.85 μL, 0.03692 mmol). After stirred under argon at −40° C. to −30° C. for 30 minutes, the reaction was quenched with methanol (0.2 mL), then treated with a methanolic sodium hydroxide solution (6N in methanol, 30.7 μL) and stirred at room temperature for another 40 minutes. The resultant mixture was poured into ethyl acetate (20 mL), naturalized with 1N HCl solution. The organic phase was washed with brine (2×5 mL), dried over magnesium sulfate, purified by PTLC (5-10% methanol in dichloromethane), to give (2′R, 3′R)-compound 8. When X=O and Y=OH, the yield was 70.9%. ¹H NMR (CDCl₃) δ7.82 (dd, J=8.22, 1.76 Hz, 1H), 7.25 (dd, J=8.38, 1.89 Hz, 1H), 7.20 (dd, J=8.07, 7.45 Hz, 1H), 6.37 (s, 1H), 4.92 (dd, J=9.71, 7.24 Hz, 1H), 3.97 (s, 3H), 3.50 (dd, J=15.40, 9.49 Hz, 1H)), 3.31 (dd, J=15.07, 7.19 Hz, 1H), 2.99 (d, J=4.79 Hz, 1H), 2.73 (d, J=4.48 Hz, 1H), 1.44 (s, 3H). MS m/e 341 (M+H⁺), 271.

### Example 9

### Preparation of Compound 9a

### 2′R,3′R

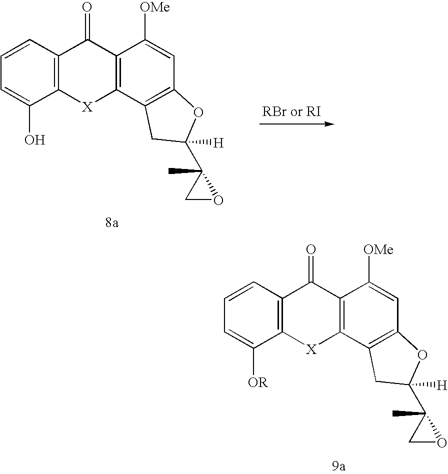

To a solution of (2′R, 3′R)-compound 8a (where Y is OH) (1.0 mg) and potassium carbonate (5 equiv) in acetone (0.5 mL), alkyl bromide or iodide (3 equiv) was added. After the mixture was refluxed for 6 hours and cooled to room temperature, the potassium salts were filtered off. The filtrate was concentrated, purified by PTLC (40-50% acetone in hexanes), to give (2′R, 3′R)-compound 9 in more than 90% yield. When X=O, R=CH₃, ¹H NMR (CDCl₃) δ 7.87 (d, J=8.51 Hz, 1H), 7.24 dd, J=8.39, 7.88 Hz, 1H), 7.15 (dd, J=8.21, 1.37 Hz, 1H). 6.37 (s, 1H), 4.87 (dd, J=10.10, 7.25 Hz, 1H), 3.99 (s, 3H), 3.97 (s, 3H), 3.54 (dd, J=15.45, 10.19 Hz, 1H), 3.55 (dd, J=15.37, 7.23 Hz, 1H), 2.97 (d, J=4.57 Hz, 1H), 2.73 (d, J=4.86 Hz, 1H), 1.44 (s, 3H). MS m/e 355 (M+H⁺), 285, 229. When X=O, R=Bn, ¹H NMR (CDCl₃) δ 7.89 (dd, J=7.19, 3.01 Hz, 1H), 7.51 (d, J=7.63 Hz, 2H), 7.41 (dd, J=7.28, 7.37 Hz, 2H), 7.35 (dd, J=7.24, 7.18 Hz, 1H), 7.22-7.18 (m, 2H), 6.37 (s, 1H), 5.26 (s, 2H), 4.86 (dd, J=9.82, 7.27 Hz, 1H), 3.96 (s, 3H), 3.49 (dd, J=15.39, 10.23 Hz, 1H), 3.29 (dd, J=15.46, 7.23 Hz, 1H), 2.97 (d, J=4.54 Hz, 1H), 2.73 (d, J=5.06 Hz, 1H), 1.43 (s, 3H). MS m/e 431 (M+H⁺), 340.

### Example 10

### Preparation of Compound 11

### 3′R

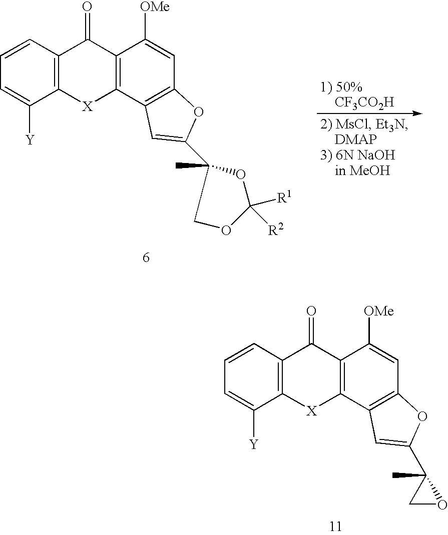

A solution of (3′R)-compound 6 (0.00923 mmol) in 50% trifluoroacetic acid in water (0.8 mL) was stirred at room temperature for 40 minutes, and then concentrated in vacuo to give the crude diol, which was used without further purification.

The crude diol and a catalytic amount of DMAP (1 mg) was dissolved in anhydrous dichloromethane (1.0 mL) and cooled to −40° C. Triethyl amine (12.7 μL, 0.0923 mmol) was added into the solution, followed by the slow addition of methanesulfonyl chloride (2.85 μL, 0.03692 mmol). After stirred under argon at −40° C. to −30° C. for 30 minutes, the solution was quenched with methanol (0.2 mL), then treated with a methanolic sodium hydroxide solution (6N in methanol, 30.7 μL) and stirred at room temperature for another 40 minutes. The resultant mixture was poured into ethyl acetate (20 mL), naturalized with 1N HCl solution. The organic phase was washed with brine (2×5 mL), dried over magnesium sulfate, purified by PTLC, to give (3′R)-compound 11. When X=O and Y=OH, the yield was 50.6%. ¹H NMR (CDCl₃) δ 9.23-9.15 (br, 1H, OH), 7.70 (dd, J=7.15, 1.98 Hz, 1H), 7.31 (dd, J=8.29, 1.91 Hz, 1H), 7.27 (dd, J=7.78, 7.61 Hz, 1H), 7.18 (ss, 2H), 4.02 (s, 3H), 3.32 (d, J=4.50 Hz, 1H), 2.96 (d, J=4.50 Hz, 1H). MS m/e 339 (M+H⁺), 324, 277, 212.

### Example 11

### Preparation of Compound 12

### 2R,5R

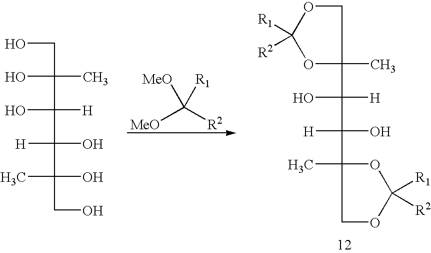

To a solution of (2R,5R)-2,5-dimethylmannitol (3.54 g, 16.8357 mmol) prepared from the reported procedures (3.54 g, 16.8357 mmol) in anhydrous DMF (6 mL) and dichloromethane (40 mL), dimethyl ketal (2.2 equiv, 37.0385 mmol) was added, followed by a slowly addition of tetrafluorobric acid (0.41 mL, 3.367 mmol). After stirred under argon at room temperature for 30 minutes, the reaction was quenched with triethylamine (0.8 mL). The resultant mixture was concentrated and isolated by chromatography on silica gel eluting with 20% ethyl acetate in hexanes, to give (2R,5R)-compound 12 in 50-70% yield. When R¹ and R²=—(CH₂)₅—, ¹H NMR (CDCl₃) δ 4.14 (d, J=9.34 Hz, 2H), 3.73 (d, J=8.86 Hz, 2H), 3.71 (d, J=4.54 Hz, 2H), 1.74-1.51 (m, 8H), 1.48-1.30 (m, 2H), 1.33 (s, 6H).

### Example 12

### Preparation of Compound 13

### 2R

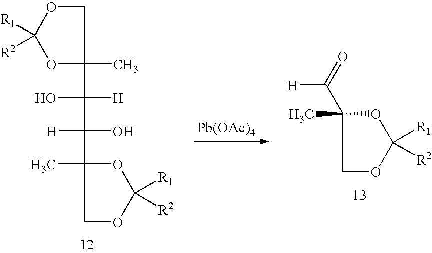

To a solution of (2R,5R)-compound 12 (9.82 mmol), and sodium carbonate (5.20 g, 49.1 mmol) in anhydrous dichloromethane (5 0 mL), lead (IV) acetate (5.38 g, 11.78 mmol) was added. After stirred under argon at room temperature for 30 minutes, the reaction mixture was poured into ethyl ether (400 mL), washed with a saturated solution of sodium carbonate (50 mL) and brine (2×30 mL). The organic phase was dried over anhydrous magnesium sulfate, concentrated and isolated by chromatography on silica gel eluting with 20% ethyl ether in dichloromethans, to give (2R)-compound 13 in 60-80% yield. When R¹ and R²=—(CH₂)₅—, ¹H NMR (CDCl₃) δ 9.65 (s, 1H), 4.23 (d, J=8.53 Hz, 1H), 3.73 (d, J=9.09 Hz, 1H), 1.70-1.54 (m, 8H), 1.49-1.36 (m, 2H), 1.35 (s, 3H).

### Example 13

### Preparation of Compound 10

### 2R

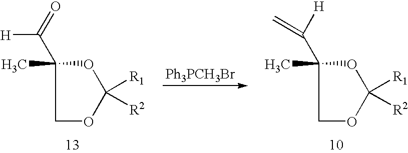

To a solution of methyltriphenylphosphonium bromide (9.458 g, 26.4784 mmol) and HMPA (800 μL) in anhydrous THF (80 mL) at −78° C., a solution of n-butyllithium (1.6 M in hexanes, 18.20 mL, 29.1262 mmol) was slowly added under argon. The reaction mixture was stirred at 0° C. for 1 hour and at room temperature for another 0.5 hour, a clear red-orange solution was generated. The resultant red-orange solution was cooled to −78° C. and added into a pro-cooled (2R)-compound 13 (13.2392 mmol) at −78° C. under argon via cannula. The reaction mixture was allowed slowly to warm to room temperature and stirred at room temperature for 2 hours. After the reaction was quenched with a saturated solution of ammonium chloride (3 mL), the formed precipitant was filtered off, the filtrate was diluted with ethyl ether (300 mL), washed with water (30 mL), brine (2×40 mL). The organic phase was dried over anhydrous magnesium sulfate, concentrated and isolated by chromatography on silica gel eluting with 15% ethyl ether in dichloromethans, to give 2R)-compound 10 in 70-90% yield. When R¹ and R²=—(CH₂)₅—, ¹H NMR (CDCl₃ δ 5.93 (dd, J=16.82, 10.35 Hz, 1H), 5.30 (d, J=18.01 Hz, 1H), 5.08 (d, J=9.80 Hz, 1H), 3.84 (d, J=8.29 Hz, 1H), 3.77 (d, J=8.28 Hz, 1H), 1.72-1.54 (m, 8H), 1.48-1.39 (m, 1H), 1.39-1.30 (m, 1H), 1.37 (s, 3H).

What is claimed is:

1. A process comprising: 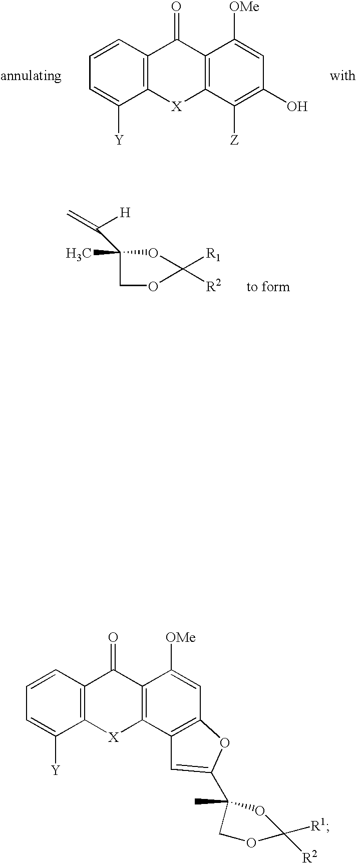
wherein X is O, S, NH, or NR;
wherein Y is H, OH, OR, R, OCOR, Cl, F, or NHSO₂CH₃;
wherein R is C₁-C₁₀ hydrocarbyl,
wherein Z is halogen; and
wherein each of R¹ and R² is independently H or C₁-C₁₀ hydrocarbyl, or R¹ and R² join together to form a C₅-C₇ ring.

2. The process defined in claim 1, the annulating step is performed using a transition metal catalyst.

3. The process defined in claim 1, further comprising before said annulating step, selectively halogenating

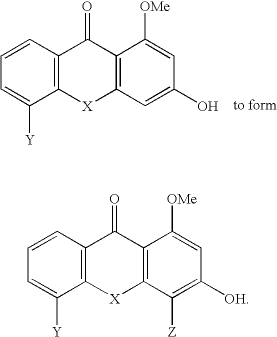

4. The process defined in claim 3 wherein the halogenating is perfomed with an elemental or complexed halogen.

5. The process defined in claim 3, further comprising before said halogenating step, condensing 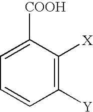
with phloroglucinol to form 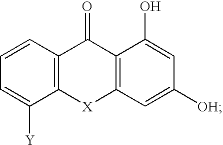
selectively protecting 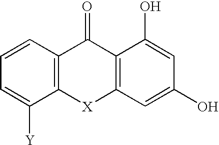
by adding benzyl halide to form 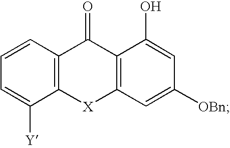
and methylating 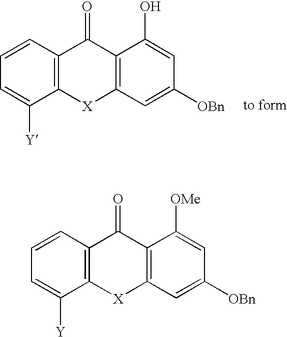
wherein Y′ is either Y or a protected OH group.

6. The process defined in claim 5, wherein the condensing step is performed in a phosphorosoxychloride solvent with a Lewis acid catalyst; wherein the protecting step is performed using benzyl halide wherein the protected Y group is O-benzyl; and the methylating step is performed using methyl iodide.

7. The process defined in claim 1, further comprising
reducing 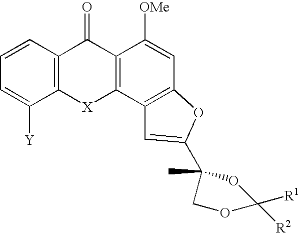
to form 2′R,3′R and 2′S,3′R diastereomers of 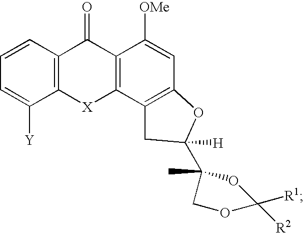
and
separating the diastereomers;
wherein each of R¹ and R² is independently H or C₁-C₆ hydrocarbyl, or R¹ and R² join together to form a C₅-C₇ hydrocarbyl ring.

8. The process defined in claim 7, wherein the reducing step is performed using hydrogen atmosphere and a transition metal catalyst; and the separating step is performed by crystallization or chromatography.

9. The process defined in claim 8 further comprising
deprotecting at least one of the separated diastereomers.

10. The process defined in claim 9 further comprising
cyclizing to an epoxide at least one of the separated diastereomers to form 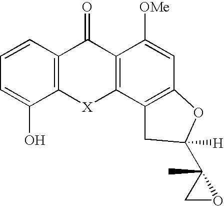
or the 2′S,3′R form thereof.

11. The process defined in claim 10 wherein the cyclizing step includes the sub steps of
activating a hydroxyl group of 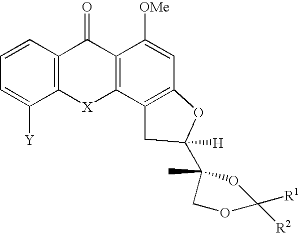
or the 2′S,3′R form thereof with a mesylate, and
cyclizing under basic conditions.

12. The process defined in claim 10 further comprising
alkylating 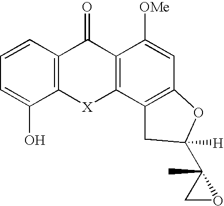
or the 2′S,3′R form thereof to form 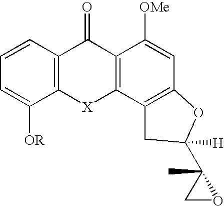
or the 2′S,3′R form thereof.

13. The process defined in claim 12 wherein the alkylating step is performed using an alkyl halide.

14. A process comprising
epoxidizing 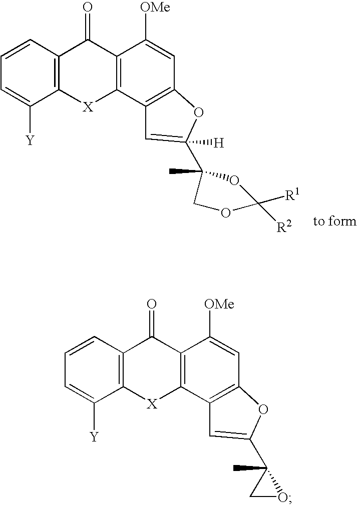
wherein each of R¹ and R² independently is H or C₁-C₆ hydrocarbyl, or R¹ and R² join together to form a C₅-C₇ hydrocarbyl ring.

15. The process defined in claim 14, wherein the cyclizing step includes the sub steps of
forming a diol on 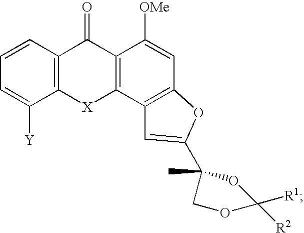
activating the hydroxyl groups of the diol with a mesylate; and
cyclizing under basic conditions.

16. A process comprising
alkylating 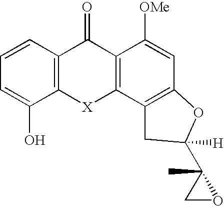
or the 2′S,3′R form thereof to form 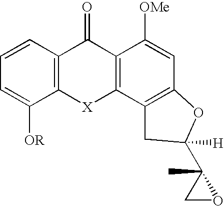
or the 2′S,3′R form thereof;
wherein X is O, S, NH, or NR; and
wherein R is C₁-C₁₀ hydrocarbyl.

17. A process comprising
cyclizing to an epoxide at least one of 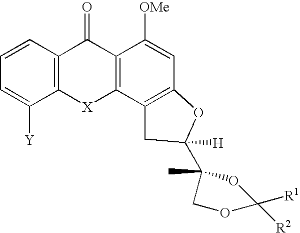
or the or 2′S,3′R form thereof to form 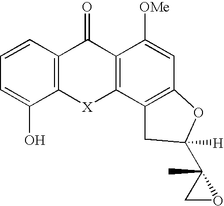
or the 2′S,3′R form thereof;
wherein each of R¹ and R² independently is H or C₁-C₆ hydrocarbyl, or R¹ and R² join together to form a C₅-C₇ hydrocarbyl ring;
wherein Y is H, OH, OR, R, Cl, F, or NHSO₂CH₃; and
wherein R is C₁-C₁₀ hydrocarbyl.

18. The process defined in claim 17, further comprising before said cyclizing step, reducing 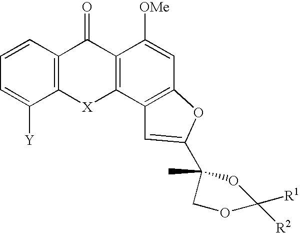
to form 2′R,3′R and 2′S,3′R diastereomers of 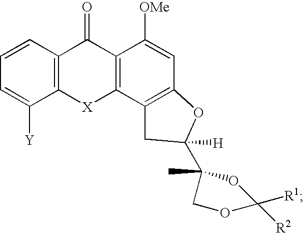
and
separating the diastereomers.

19. The process defined in claim 18, further comprising before said reducing step [chemical structure]
